(19) United States
(12) Patent Application Publication
(10) Pub. No.: US 2025/0230126 A1
(43) Pub. Date: Jul. 17, 2025

(54) INTERMEDIATE OF POLYAMINE DERIVATIVE, PREPARATION METHOD THEREFOR, AND USE THEREOF

(71) Applicant: WUHAN WUYAO SCI&TECH CO., LTD, Wuhan, Hubei (CN)

(72) Inventors: Yishun XIA, Wuhan, Hubei (CN); Xiang RAO, Wuhan, Hubei (CN); Chao CAI, Wuhan, Hubei (CN); Xinrong LIU, Wuhan, Hubei (CN); Lei LI, Wuhan, Hubei (CN); Zhenwu LI, Wuhan, Hubei (CN); Bo YANG, Wuhan, Hubei (CN)

(21) Appl. No.: 18/701,524

(22) PCT Filed: Nov. 2, 2022
(86) PCT No.: PCT/CN2022/129147

§ 371 Date: Apr. 15, 2024

(30) Foreign Application Priority Data
Nov. 3, 2021 (CN) 202111310146.2

(51) Int. Cl. C07D 207/404; C07C 219/22; C07C 231/12; C07C 327/16

(52) U.S. Cl. CPC C07D 207/404; C07C 219/22; C07C 231/12; C07C 327/16

(57) ABSTRACT

An intermediate of a polyamine derivative, a preparation method therefor, and a use thereof. The intermediate is easy to prepare and high in purity; and when the intermediate is used for preparing the polyamine derivative, the obtained product is high in purity, the operation is simple and convenient, the purity and yield of the polyamine derivative are improved, the purification operation for the product is simplified, and the industrial production of the polyamine derivative and a medicinal salt thereof can be improved. Further provided is a preparation method for the polyamine derivative, the operation thereof is simple and convenient, and the purity of the obtained product is high. The method has a very good application prospect in the field of chemical medicines.

### FIELD OF THE INVENTION

The present invention relates to the technical field of chemical medicines, and in particular to an intermediate of a polyamine derivative, a preparation method therefor, and use thereof.

### BACKGROUND OF THE INVENTION

Systemic inflammatory response syndrome and autoimmune disorder-related diseases, such as sepsis and autoimmune diseases, are two categories of diseases caused by the body's own excessive immune response. Currently, there is still a lack of effective therapeutic drugs, and the targeted prevention and treatment of these diseases are the focus and hot issues of clinical concern. Sepsis refers to the systemic inflammatory response syndrome (SIRS) caused by infection. Septic shock is a severe stage of sepsis and is one of the main factors causing patient mortality in ICU.

Studies show that the pathogenesis of sepsis lies in the fact that pathogen-associated molecular patterns (PAMPs) released by bacteria, viruses, fungi, and other pathogens are recognized by pattern recognition receptors (PRRs) of the host's innate immune system, mediating the activation of inflammatory response cells and thus causing a systemic excessive inflammatory response. Epidemiological surveys show that PAMP molecules that cause sepsis mainly include bacterial lipopolysaccharide (LPS), bacterial genomic DNA (CpG DNA), peptidoglycan (PGN), lipoteichoic acid (LTA), viral RNA, and zymosan.

Chinese patent No. CN105348137B discloses a pharmaceutically acceptable salt of a polyamine derivative, a preparation method therefor, and use thereof, wherein the pharmaceutically acceptable salt of the polyamine derivative can be used in the preparation of a medicament for treating sepsis; however, in the preparation process, the purity of the intermediates and final product was not considered, so that the preparation method needs to be further improved.

Therefore, it is very necessary to develop a preparation method suitable for industrialization that can obtain a polyamine derivative and an intermediate thereof with high purity.

### SUMMARY OF THE INVENTION

In order to overcome the defects of the prior art, the present invention provides a preparation method for a polyamine derivative and an intermediate thereof and use of the polyamine derivative and the intermediate thereof.

In a first aspect of the present invention, provided is a compound of formula I, having the following structure:

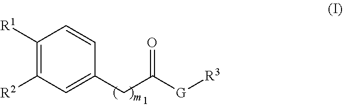

wherein
m₁ is selected from an integer of 1-6 (e.g., 1, 2, 3, 4, 5, or 6);
R¹ and R² are independently selected from: OH and alkoxy;
G is selected from O and S;
R³ is selected from:

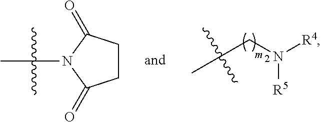

wherein m₂ is selected from an integer of 1-6 (e.g. 1, 2, 3, 4, 5, or 6), and R⁴ and R⁵ are independently selected from alkyl.

Specifically, m₁ is selected from an integer of 1-3, such as 1, 2, or 3, particularly 2.

Specifically, m₂ is selected from an integer of 1-3, such as 1, 2, or 3, particularly 2.

Specifically, R¹ and R² are independently selected from: OH and C₁-C₆ alkoxy; more specifically, R¹ and R² are independently selected from: OH and C₁-C₃ alkoxy; and further specifically, R¹ and R² are independently selected from: OH, methoxy, and ethoxy.

Specifically, the compound of formula I may have the following structure:

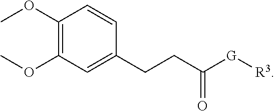

Specifically, R⁴ and R⁵ are independently selected from C₁-C₆ alkyl, particularly C₁-C₃ alkyl, such as methyl, ethyl, and n-propyl.

In some embodiments of the present invention, R³ is selected from:

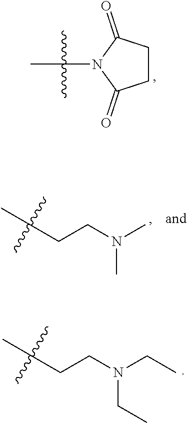

In some embodiments of the present invention, the compound of formula I has the following structure:

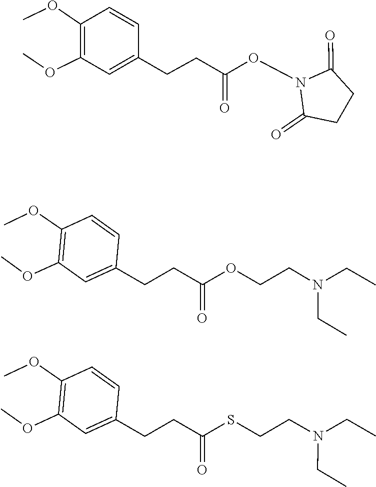

In a second aspect of the present invention, provided is a preparation method for the compound of formula I, comprising a step of reacting a compound of formula II with R³-G-H:

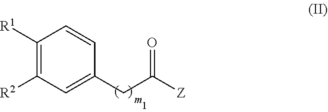

wherein Z is halogen, particularly chlorine; and m₁ and R¹-R² are as defined as in the compound of formula I described above;
in the R³-G-H, G and R³ are as defined as in the compound of formula I described above.

Specifically, the reaction is carried out under a protective gas, wherein the protective gas may be an inert gas, such as nitrogen or argon, preferably nitrogen.

Specifically, the reaction is carried out at a temperature of 15 to 35° C. (e.g., 15, 16, 18, 20, 22, 24, 25, 26, 28, 30, 32, 34 or 35° C.), particularly a temperature of 20-30° C.

Specifically, the reaction may be carried out for a period of 1-6 h (e.g., 1, 2, 3, 4, 5 or 6 h), particularly a period of 2-4 h.

Specifically, the reaction is carried out in a solvent, wherein the solvent is selected from: dichloromethane, chloroform, ethyl acetate, n-hexane, cyclohexane, and methyl tert-butyl ether, particularly dichloromethane.

Specifically, the reaction system further comprises an acid scavenger, such as an organic weak base and an inorganic weak base, which can be selected from pyridine, triethylamine, N,N-diisopropylethylamine, an alkali metal acetate (e.g., sodium acetate), and/or an alkali metal carbonate (e.g., sodium carbonate or potassium carbonate), preferably triethylamine.

Specifically, the preparation method further comprises a step of purifying the reaction product.

Specifically, the purification step comprises one or more selected from: washing, concentrating, crystallizing, filtering, and drying.

Specifically, the preparation method comprises: under a protective gas, mixing R³-G-H with
a solvent, dropwise adding an acid scavenger, adding the compound of formula II during the dropwise addition, and reacting; and
after the reaction is completed, purifying the reaction product.

Specifically, the acid scavenger is dropwise added at a temperature of −10 to 10° C. (e.g., −10, −8, −6, −5, −4, −2, 0, 2, 4, 5, 6, 8 or 10° C.), particularly at a temperature of at −5 to 5° C.

In a third aspect of the present invention, provided is a preparation method for a compound of formula III:

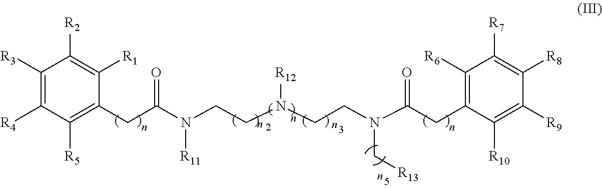

wherein
R₁-R₁₀ are independently selected from: H, OH, alkoxy, aryloxy, and aralkoxy;
R₁₁ is H, an amino protecting group, or

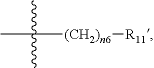

wherein R₁₁′ is —CN, —CH₂—NH₂,
or —CH₂—NH—R₁₁″, and R₁₁″ is an amino protecting group;
R₁₂ is H, an amino protecting group, or

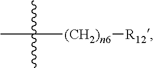

wherein R₁₂′ is —CN, —CH₂—NH₂, or —CH₂—NH—R₁₂″, and R₁₂″ is an amino protecting group;
R₁₃ is —CN, —CH₂—NH₂, or —CH₂—NH—R₁₃′, and R₁₃′ is an amino protecting group;
n₁-n₇ are independently selected from an integer of 0-10 (e.g., 0, 1, 2, 3, 4, 5, 6, 7, 8, 9, or 10);
n′ is 0 or 1;
the preparation method comprises a step (a) of reacting a compound of formula IV with a compound of formula V and/or a compound of formula VI:

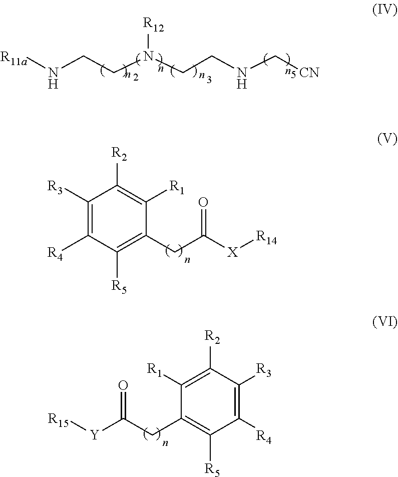

wherein R₁₁ₐ is H, an amino protecting group, or

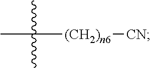

R₁₂ₐ is H, an amino protecting group, or

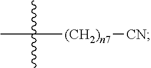

X and Y are independently selected from O and S;
R₁₄ and R₁₅ are independently selected from

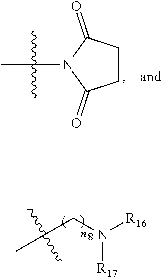

wherein n₈ is an integer of 1-6 (e.g., 1, 2, 3, 4, 5, or 6), and R₁₆ and R₁₇ are independently selected from alkyl;
and n₁-n₇, n′, and R₁-R₁₀ are as defined as in the compound of formula III.

Specifically, n₈ is an integer of 1-3, such as 1, 2, or 3, particularly 2.

Specifically, R₁₆ and R₁₇ are independently selected from C₁-C₆ alkyl, particularly C₁-C₃ alkyl, such as methyl, ethyl, and n-propyl.

In some embodiments of the present invention, R₁₄ and R₁₅ are independently selected from:

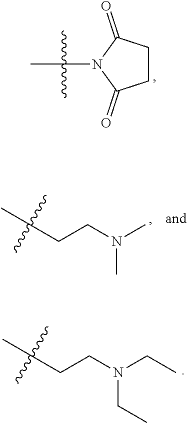

In some embodiments of the present invention, R₁₁ₐ is

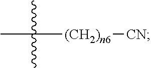

In some embodiments of the present invention, R₁₂ₐ is an amino protecting group.

Specifically, the reaction described above is carried out under a protective gas, wherein the protective gas may be an inert gas, such as nitrogen or argon, preferably nitrogen.

Specifically, the reaction described above is carried out at a temperature of 50-70° C. (e.g., 50, 52, 54, 55, 56, 58, 60, 62, 64, 65, 66, 68 or 70° C.), particularly a temperature of 55-65° C.

Specifically, the reaction described above is carried out for a period of 6-60 h (e.g., 6, 12, 24, 30, 36, 40, 42, 48, 54 or 60 h), particularly a period of 40-48 h.

Specifically, the reaction described above is carried out in a solvent, wherein the solvent can be selected from: ethyl acetate, isopropyl acetate, acetonitrile, dichloromethane, tetrahydrofuran, toluene, xylene, chlorobenzene, dioxane, and the like; particularly, the solvent is ethyl acetate, toluene, or dioxane, preferably ethyl acetate.

Specifically, the preparation method further comprises a step of purifying the reaction product.

Specifically, the purification step may comprise washing the reaction product, wherein a solvent for the washing may be water.

Specifically, the purification step may further comprise concentrating the washed reaction product. More specifically, the preparation method comprises: under a protective gas, mixing the compound of formula IV with a reaction solvent, heating, adding the compound of formula V and/or the compound of formula VI, and reacting; and
after the reaction is completed, purifying the reaction product.

Specifically, n₁ is an integer of 1-5, such as an integer of 1-3, for example, 2.

Specifically, n₂ is an integer of 0-5, such as an integer of 1-3, for example, 2.

Specifically, n₃ is an integer of 0-5, such as an integer of 1-3, for example, 2.

Specifically, n₄ is an integer of 1-5, such as an integer of 1-3, for example, 2.

Specifically, n₅ is an integer of 0-5, such as an integer of 1-3, for example, 2.

Specifically, n₆ is an integer of 0-5, such as an integer of 1-3, for example, 2.

Specifically, n₇ is an integer of 0-5, such as an integer of 1-3, for example, 2.

In some embodiments of the present invention, n′ is 1.

Specifically, the compound of formula IV has the following structure:

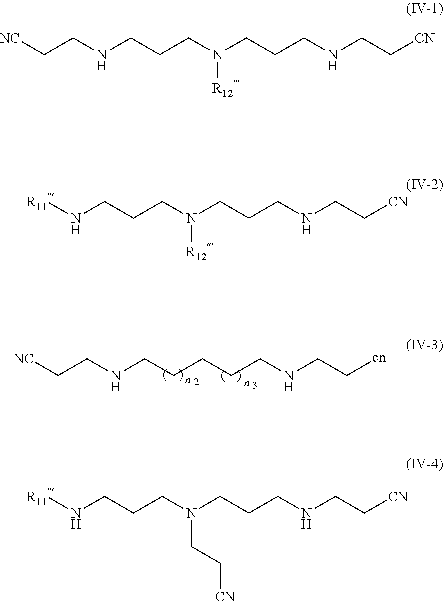

wherein R₁₁′″ and R₁₂′″ are independently selected from an amino protecting group.

Specifically, the compound of formula V has the following structure:

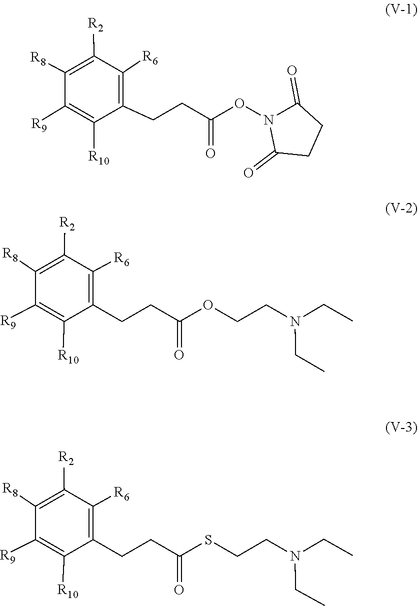

Specifically, the compound of formula VI has the following structure:

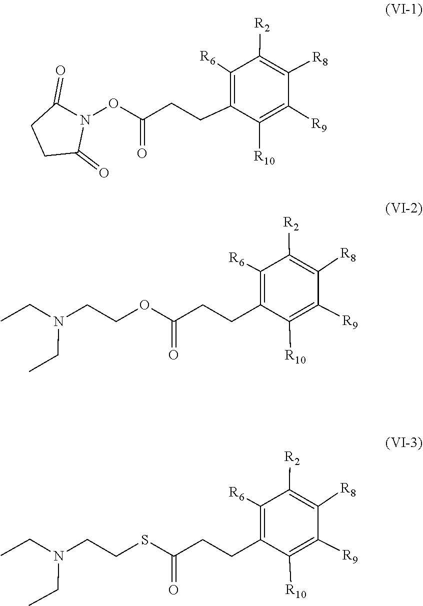

Specifically, R₁ is H.

Specifically, R₄ is H.

Specifically, R₅ is H.

Specifically, R₆ is H.

Specifically, R₉ is H.

Specifically, R₁₀ is H.

Specifically, R₂, R₃, R₇, and R₈ are independently selected from: H, OH, alkoxy, aryloxy, and aralkoxy; more specifically, R₂, R₃, R₇, and R₈ are independently selected from: H, OH, C₁-C₆ alkoxy, C₆-C₁₂ aryloxy, and C₇-C₁₂ aralkoxy; further specifically, R₂, R₃, R₇, and R₈ are independently selected from: H, OH, and C₁-C₆ alkoxy; and still further specifically, R₂, R₃, R₇, and R₈ are independently selected from: OH, methoxy, and ethoxy.

Specifically, R₂ and R₃ are the same, and/or R₇ and R₈ are the same; and preferably, R₂, R₃, R₇, and R₈ are all the same.

In some embodiments of the present invention, the compound of formula V has the same structure as the compound of formula VI.

In some embodiments of the present invention, the preparation method described above comprises the step of reacting the compound of formula IV with the compound of formula V, wherein the compound of formula IV has a structure of formula IV-1, and the compound of formula V has a structure of V-1, V-2 or V-3.

In some embodiments of the present invention, in formula III, R₁₁ is

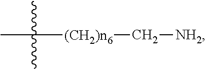

R₁₂ is an amino protecting group, and R₁₃ is —CH₂—NH₂; the preparation method further comprises step (b): reducing a reaction product of step (a).

Specifically, the reduction reaction system may further comprise a solvent such as an alcohol, for example, ethanol, isopropanol, or tert-butanol, particularly ethanol.

Specifically, the reduction reaction system further comprises a catalyst such as Raney Ni, palladium on carbon, or the like.

In other embodiments of the present invention, in formula III, R₁₁ is

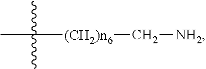

R₁₂ is H, and R₁₃ is —CH₂—NH₂; the preparation method further comprises step (c): subjecting the reduced product of step (b) to an amino deprotection treatment.

In some embodiments of the present invention, the preparation method described above comprises the step of reacting the compound of formula IV with the compound of formula V, wherein the compound of formula IV has a structure of formula IV-1, and the compound of formula V is selected from the following structures:

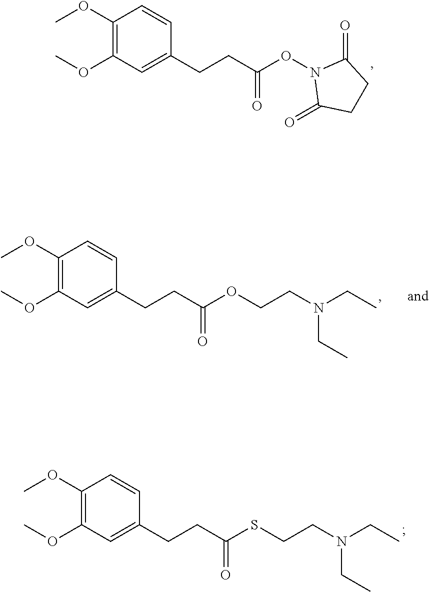

the compound of formula I obtained has the following structure:

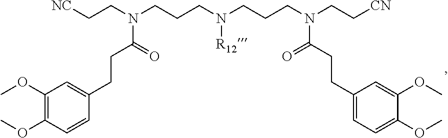

which can be further reduced to prepare

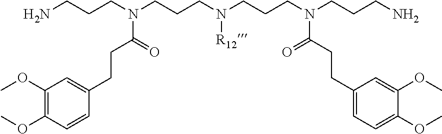

and then further deprotected to prepare

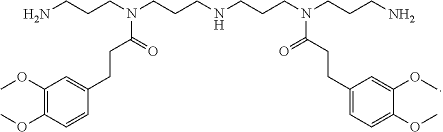

In a fourth aspect of the present invention, provided is use of the compound according to the first aspect in the preparation of a polyamine derivative or a pharmaceutically acceptable salt thereof. Specifically, the polyamine derivative has the following structure:

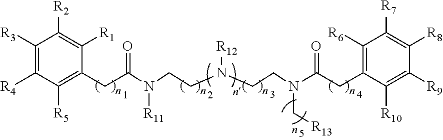

wherein
R₁-R₁₀ are independently selected from: H, OH, alkoxy, aryloxy, and aralkoxy;
R₁₁ is H of

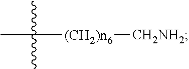

R₁₂ is H or

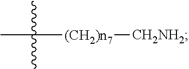

R₁₃ is —CH₂—NH₂; and
n₁-n₇ are independently selected from an integer of 0-10 (e.g., 0, 1, 2, 3, 4, 5, 6, 7, 8, 9, or 10);
n′ is 0 or 1.

Specifically, n₁ is an integer of 1-5, such as an integer of 1-3, for example, 2.

Specifically, n₂ is an integer of 0-5, such as an integer of 1-3, for example, 2.

Specifically, n₃ is an integer of 0-5, such as an integer of 1-3, for example, 2.

Specifically, n₄ is an integer of 1-5, such as an integer of 1-3, for example, 2.

Specifically, n₅ is an integer of 0-5, such as an integer of 1-3, for example, 2.

Specifically, n₆ is an integer of 0-5, such as an integer of 1-3, for example, 2.

Specifically, n₇ is an integer of 0-5, such as an integer of 1-3, for example, 2.

In some embodiments of the present invention, n′ is 1.

Specifically, R₁ is H.

Specifically, R₄ is H.

Specifically, R₅ is H.

Specifically, R₆ is H.

Specifically, R₉ is H.

Specifically, R₁₀ is H.

Specifically, R₂, R₃, R₇, and R₈ are independently selected from: H, OH, alkoxy, aryloxy, and aralkoxy; more specifically, R₂, R₃, R₇, and R₈ are independently selected from: H, OH, C₁-C₆ alkoxy, C₆-C₁₂ aryloxy, and C₇-C₁₂ aralkoxy; further specifically, R₂, R₃, R₇, and R₈ are independently selected from: H, OH, and C₁-C₆ alkoxy; and still further specifically, R₂, R₃, R₇, and R₈ are independently selected from: OH, methoxy, and ethoxy.

Specifically, R₂ and R₃ are the same, and/or R₇ and R₈ are the same; preferably, R₂, R₃, R₇, and R₈ are all the same, for example, all are methoxy.

In some embodiments of the present invention, R₁₁ is

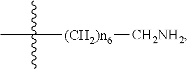

### and R₁₂ is H.

In one embodiment of the present invention, the polyamine derivative has the following structure:

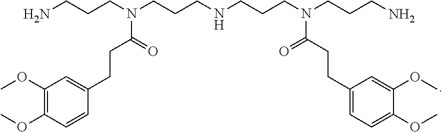

In a fifth aspect of the present invention, provided is use of the compound according to the first aspect in the preparation of a medicament for antagonizing bacterial lipopolysaccharide (LPS), bacterial genomic DNA (CpG DNA), peptidoglycan (PGN), lipoteichoic acid (LTA), viral RNA, and zymosan.

In a sixth aspect of the present invention, provided is use of the compound according to the first aspect in the preparation of a medicament for treating sepsis.

The present invention provides an intermediate of a polyamine derivative, a preparation method therefor, and use thereof, wherein the intermediate is easy to prepare and has a high purity; when the intermediate is used for preparing the polyamine derivative, it is beneficial to improving the purity and yield of the product, and the operation is simple and convenient, which is beneficial to the purity and yield of the polyamine derivative and simplification of the purification process for the product, thereby facilitating the improvement of the industrial production of the polyamine derivative and a pharmaceutically acceptable salt thereof. The present invention further provides a preparation method for the polyamine derivative, which is simple and convenient to operate, yields a product with high purity, and thus has very good application prospects in the field of chemical medicines.

### BRIEF DESCRIPTION OF THE DRAWING

FIG. 1 shows a nuclear magnetic resonance hydrogen spectrum of compound 1a prepared in the example of the present invention. Instrument model: Bruker avance 400 (400 MHz) nuclear magnetic resonance spectrometer; test condition: 400 MHz; solvent: deuterated chloroform.

### DETAILED DESCRIPTION OF THE INVENTION

Unless otherwise defined, all scientific and technical terms used in the present invention have the same meaning as commonly understood by those skilled in the art to which the present invention relates.

The amino protecting group involved in the present invention may be any suitable known amino protecting group, such as tert-butoxycarbonyl (Boc), benzyloxycarbonyl (Cbz), methanesulfonyl (Ms), or p-toluenesulfonyl (Ts), particularly Boc.

The inert gas involved in the present invention refers to a gas that does not participate in the reaction, and may include nitrogen and the like in addition to rare gases such as helium, neon, argon, krypton, and xenon, depending on the specific reaction conditions.

The term “alkyl” refers to a hydrocarbyl group that is linear or branched and that does not contain unsaturated bonds, and that is linked to the rest of the molecule via a single bond. The alkyl used herein generally contains 1 to 12 (e.g., 1, 2, 3, 4, 5, 6, 7, 8, 9, 10, 11, or 12) carbon atoms, and preferably contains 1 to 6 carbon atoms (i.e., C₁-C₆ alkyl). Examples of alkyl include, but are not limited to, methyl, ethyl, n-propyl, isopropyl, n-butyl, isobutyl, tert-butyl, n-pentyl, isopentyl, neopentyl, tert-pentyl, n-hexyl, isohexyl, and the like.

The term “alkoxy” refers to a substituent formed from a hydroxy group by substituting the hydrogen atom with alkyl. The alkoxy used herein generally contains 1 to 12 (e.g., 1, 2, 3, 4, 5, 6, 7, 8, 9, 10, 11, or 12) carbon atoms, and preferably contains 1 to 6 carbon atoms (i.e., C₁-C₆ alkoxy). Examples of alkoxy include, but are not limited to, methoxy, ethoxy, propoxy, butoxy, and the like. The substituent formed from a hydroxy group by substituting the hydrogen atom with aryl is aryloxy, and the aryloxy used herein generally contains 6 to 18 (e.g., 6, 8, 10, 12, 14, 16, or 18) carbon atoms, and preferably contains 6 to 12 carbon atoms (i.e., C₆-C₁₂ aryloxy). Examples of the aryloxy include, but are not limited to, phenoxy. The substituent formed from an alkoxy group by substituting the hydrogen atom of a hydroxy group with aralkyl is aralkoxy, and the aralkoxy used herein generally contains 7 to 18 (e.g., 7, 8, 9, 10, 11, 12, 13, 14, 15, 16, 17, or 18) carbon atoms, and preferably contains 7 to 12 carbon atoms (i.e., C₇-C₁₂ aralkoxy). Examples of aralkoxy include, but are not limited to, benzyloxy.

The term “halogen” refers to bromine, chlorine, iodine, or fluorine, preferably chlorine.

The disclosures of the various publications, patents, and published patent specifications cited herein are hereby incorporated by reference in their entireties.

The technical solutions of the present invention will be clearly and completely described below with reference to the examples of the present invention, and it is obvious that the described examples are only a part of the examples of the present invention but not all of them. Based on the examples of the present invention, all other examples obtained by those of ordinary skills in the art without creative work shall fall within the protection scope of the present invention.

### Example 1

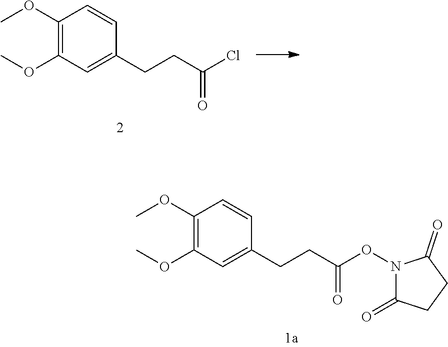 [SMILES: COc1ccc(CCC(=O)Cl)cc1OC ; COc1ccc(CCC(=O)ON2C(=O)CCC2=O)cc1OC]

Under nitrogen atmosphere, 798 g of dichloromethane was added into a glass reaction kettle, followed by the addition of 90 g (0.78 mol) of NHS (N-hydroxysuccinimide). The mixture was stirred for 5-15 min, cooled to −5 to 5° C., controlled at −5 to 5° C., and dropwise added with 79.5 g of triethylamine. During the dropwise addition, the solid was gradually dissolved to give a clear solution. Compound 2 (3,4-dimethoxyphenylpropionyl chloride) (0.85 mol, 195.6 g) was added with the temperature controlled at −5 to 5° C. A white mist was generated, and a solid was precipitated. The mixture was slowly heated to 20-30° C., stirred for reaction for 2-4 h with the temperature controlled at 20-30° C., washed, concentrated, crystallized, filtered, and dried to give 199.5 g of a solid, namely compound 1a with a purity of 99.3%.

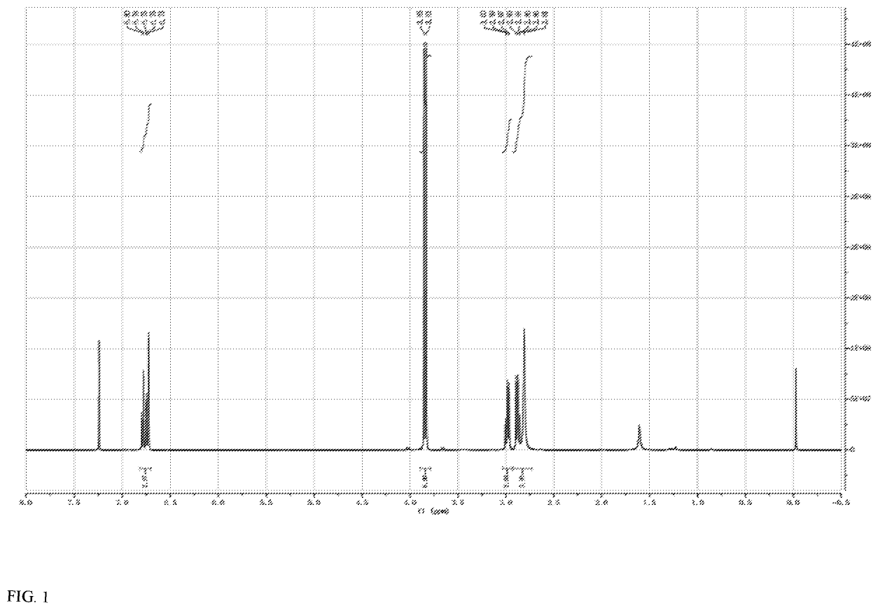

Compound 1a: Mass spectrum: m/z=307.3, and nuclear magnetic resonance hydrogen spectrum as shown in FIG. 1.

### Example 2

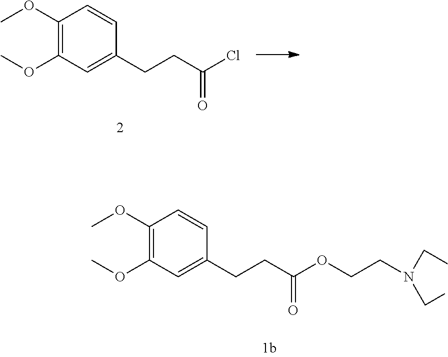 [SMILES: COc1ccc(CCC(=O)Cl)cc1OC ; CCN(CC)CCOC(=O)CCc1ccc(OC)c(OC)c1]

Under nitrogen atmosphere, 798 g of dichloromethane was added into a glass reaction kettle, followed by the addition of 92 g of 2-diethylaminoethanol (0.78 mol). The mixture was stirred for 5-15 min, cooled to −5 to 5° C., controlled at −5 to 5° C., and dropwise added with 79.5 g of triethylamine. During the dropwise addition, the solid was gradually dissolved to give a clear solution. 3,4-dimethoxyphenylpropionyl chloride (0.85 mol, 195.6 g) was added with the temperature controlled at −5 to 5° C. A white mist was generated, and a solid was precipitated. The mixture was slowly heated to 20-30° C., stirred for reaction for 2-4 h with the temperature controlled at 20-30° C., washed, concentrated, crystallized, filtered, and dried to give 170.2 g of a solid, namely compound 1b with a purity of 96.2%.

### Example 3

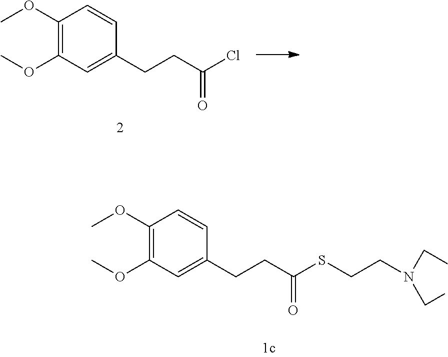 [SMILES: COc1ccc(CCC(=O)Cl)cc1OC ; CCN(CC)CCSC(=O)CCc1ccc(OC)c(OC)c1]

Under nitrogen atmosphere, 798 g of dichloromethane was added into a glass reaction kettle, followed by the addition of 103.7 g of 2-diethylaminoethanethiol (0.78 mol). The mixture was stirred for 5-15 min, cooled to −5 to 5° C., controlled at −5 to 5° C., and dropwise added with 79.5 g of triethylamine. During the dropwise addition, the solid was gradually dissolved to give a clear solution. 3,4-dimethoxyphenylpropionyl chloride (0.85 mol, 195.6 g) was added with the temperature controlled at −5 to 5° C. A white mist was generated, and a solid was precipitated. The mixture was slowly heated to 20-30° C., stirred for reaction for 2-4 h with the temperature controlled at 20-30° C., washed, concentrated, crystallized, filtered, and dried to give 165.2 g of a solid, namely compound 1c with a purity of 94.3%.

### Example 4

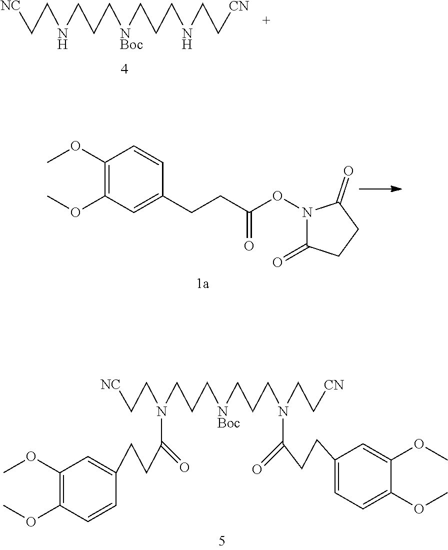

Under nitrogen atmosphere, 60.9 g (0.18 mol) of compound 4 was added into a 1000 mL three-neck flask, followed by the addition of 265.6 g of ethyl acetate. The mixture was stirred for 5-15 min, heated to 55-65° C., controlled at 55-65° C., and added with 98.6 g (0.32 mol) of compound 1a. After the addition, the mixture was stirred for reaction for another 40-48 h with the temperature controlled at 55-65° C., washed with water, and concentrated to give 115 g of compound 5 with a purity of 79.29%.

### Example 5

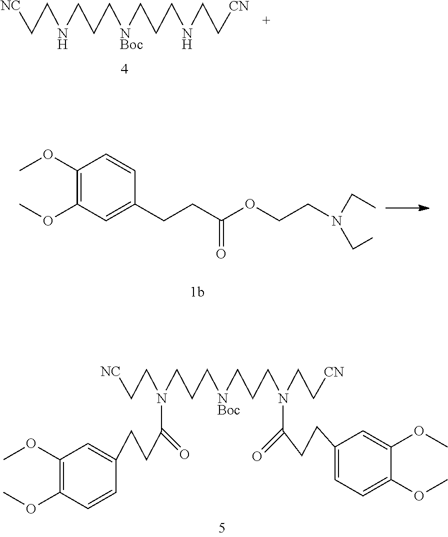

Under nitrogen atmosphere, 60.9 g (0.18 mol) of compound 4 was added into a 1000 mL three-neck flask, followed by the addition of 265.6 g of ethyl acetate. The mixture was stirred for 5-15 min, heated to 55-65° C., controlled at 55-65° C., and added with 98.9 g (0.32 mol) of compound 1b. After the addition, the mixture was stirred for reaction for another 40-48 h with the temperature controlled at 55-65° C., washed with water, and concentrated to give 103 g of compound 5 with a purity of 72.56%.

### Example 6

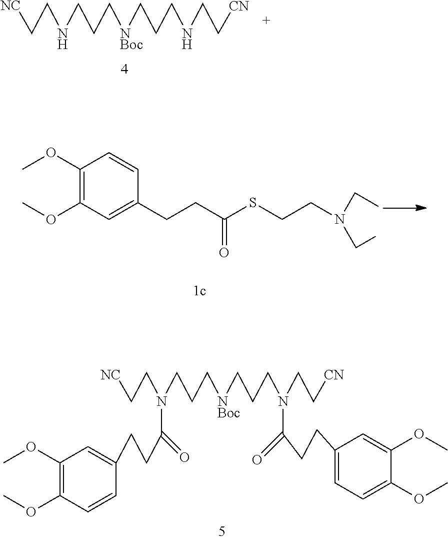

Under nitrogen atmosphere, 60.9 g (0.18 mol) of compound 4 was added into a 1000 mL three-neck flask, followed by the addition of 265.6 g of ethyl acetate. The mixture was stirred for 5-15 min, heated to 55-65° C., controlled at 55-65° C., and added with 104 g (0.32 mol) of compound 1c. After the addition, the mixture was stirred for reaction for another 40-48 h with the temperature controlled at 55-65° C., washed with water, and concentrated to give 96 g of compound 5 with a purity of 70.89%.

### Example 7

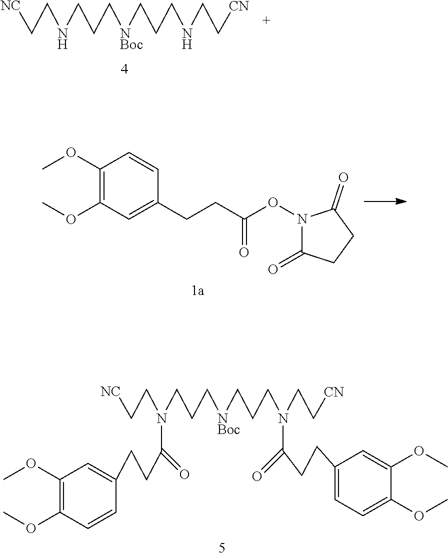

Under nitrogen atmosphere, 60.9 g (0.18 mol) of compound 4 was added into a 1000 mL three-neck flask, followed by the addition of 266 g of toluene. The mixture was stirred for 5-15 min, heated to 55-65° C., controlled at 55-65° C., and added with 98.6 g (0.32 mol) of compound 1a. After the addition, the mixture was stirred for reaction for another 40-48 h with the temperature controlled at 55-65° C., washed with water, and concentrated to give 100 g of compound 5 with a purity of 62.46%.

### Example 8

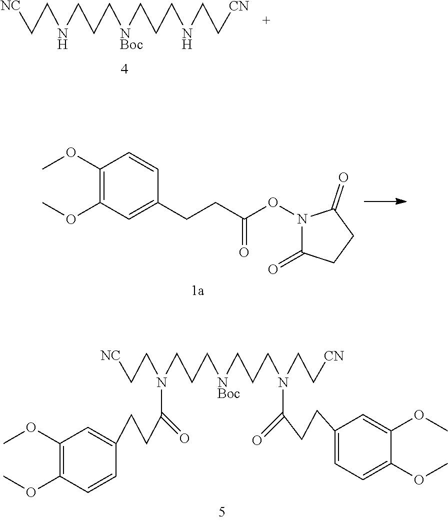

Under nitrogen atmosphere, 60.9 g (0.18 mol) of compound 4 was added into a 1000 mL three-neck flask, followed by the addition of 266 g of dioxane. The mixture was stirred for 5-15 min, heated to 55-65° C., controlled at 55-65° C., and added with 98.6 g (0.32 mol) of compound 1a. After the addition, the mixture was stirred for reaction for another 40-48 h with the temperature controlled at 55-65° C., washed with water, and concentrated to give 98 g of compound 5 with a purity of 59.38%.

### Comparative Example 1

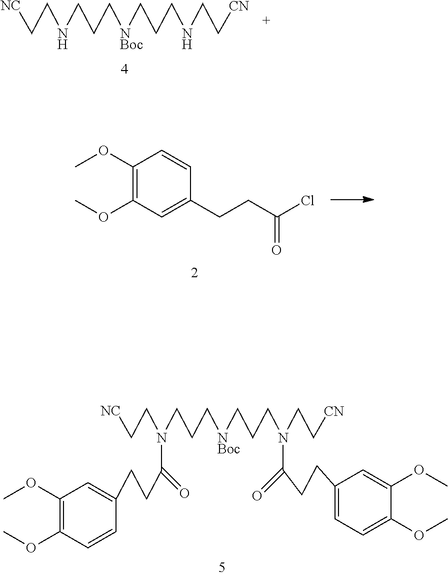

Under nitrogen atmosphere, 60.9 g (0.18 mol) of compound 4 was added into a 1000 mL three-neck flask, followed by the addition of 265.6 g of ethyl acetate. The mixture was stirred for 5-15 min, heated to 55-65° C., controlled at 55-65° C., and added with 72.8 g (0.32 mol) of compound 2. After the addition, the mixture was stirred for reaction for another 40-48 h with the temperature controlled at 55-65° C., washed with water, and concentrated to give 91 g of compound 5 with a purity of 45.71%.

### Comparative Example 2

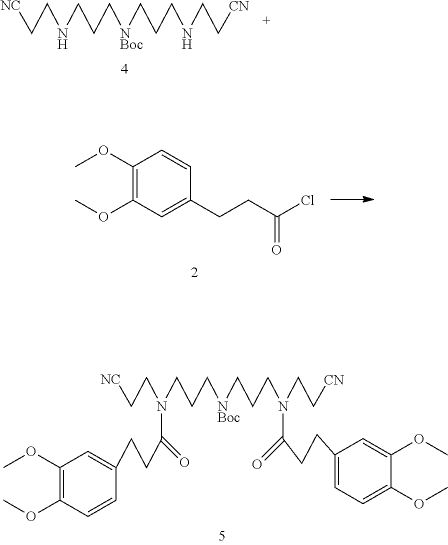

5.3 g of compound 4 was dissolved in 25 mL of dichloromethane, followed by the addition of 6 mL of triethylamine. 40 mL of a solution of compound 2 dissolved in dichloromethane (at a concentration of 10%) was dropwise added at 0° C. The mixture was allowed to react for 24 h, and concentrated to remove the organic solvent. The residue was extracted with diethyl ether, dried over anhydrous sodium sulfate, and concentrated by rotary evaporation to remove the solvent to give 7.8 g of compound 5 with a purity of 33.62%.

The above description is only for the purpose of illustrating the preferred embodiments of the present invention, and is not intended to limit the scope of the present invention. Any modifications, equivalents, and the like made without departing from the spirit and principle of the present invention shall fall in the protection scope of the present invention.

The foregoing examples and methods described herein may vary based on the abilities, experience, and preferences of those skilled in the art.

The certain order in which the steps of the method are listed in the present invention does not constitute any limitation on the order of the steps of the method.

1.-10. (canceled)

11. A compound, having a structure of formula I: 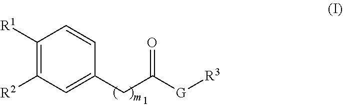
wherein
m₁ is selected from an integer of 1-6;
R¹ and R² are independently selected from: OH and alkoxy;
G is selected from O and S;
R³ is selected from: 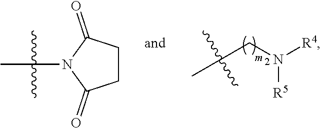
wherein m₂ is selected from an integer of 1-6, and R⁴ and R⁵ are independently selected from alkyl.

12. The compound according to claim 11, wherein the compound has the following structure:

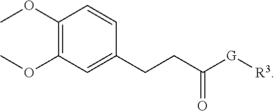

13. The compound according to claim 11, wherein R⁴ and R⁵ are independently selected from C₁-C₆ alkyl.

14. The compound according to claim 11, wherein R³ is selected from:

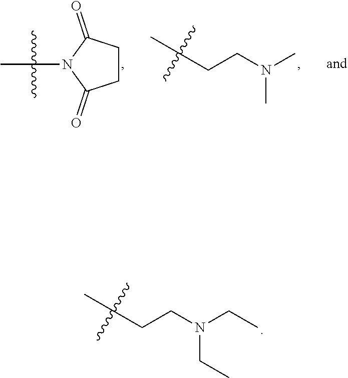

15. The compound according to claim 11, wherein the compound has the following structure:

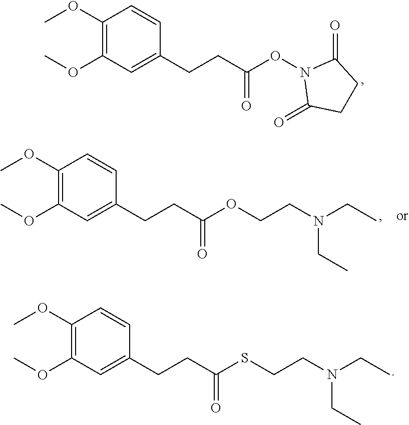

16. A preparation method for the compound according to claim 11, comprising a step of reacting a compound of formula II with R³-G-H, wherein the compound of formula II has the following structure: 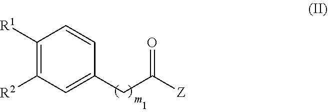
wherein Z is halogen.

17. The preparation method according to claim 16, wherein the reaction is carried out in a solvent, wherein the solvent is selected from: dichloromethane, chloroform, ethyl acetate, n-hexane, cyclohexane, and methyl tert-butyl ether.

18. The preparation method according to claim 16, wherein the reaction system further comprises an acid scavenger.

19. The preparation method according to claim 16, wherein the reaction is carried out at a temperature of 15 to 35° C.

20. Use of the compound according to claim 11 in the preparation of a polyamine derivative or a pharmaceutically acceptable salt thereof, wherein the polyamine derivative has the following structure: 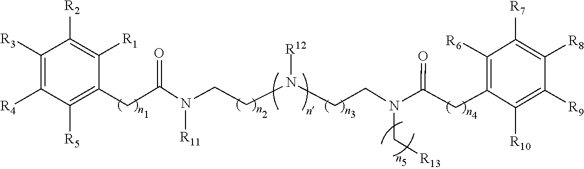
wherein
R₁-R₁₀ are independently selected from: H, OH, alkoxy, aryloxy, and aralkoxy;
R₁₁ is H or 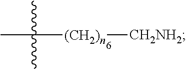
R₁₂ is H or 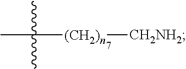
R₁₃ is —CH₂—NH₂;
n₁-n₇ are independently selected from an integer of 0-10; and
n′ is 0 or 1.

21. A preparation method for a compound of formula III, wherein the compound of formula III has the following structure: 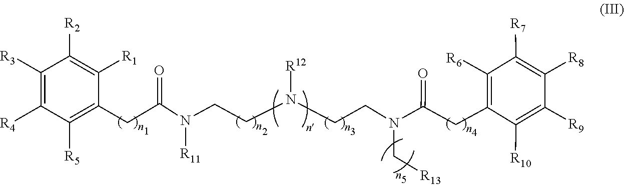
wherein
R₁-R₁₀ are independently selected from: H, OH, alkoxy, aryloxy, and aralkoxy;
R₁₁ is H, an amino protecting group, or 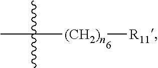
wherein R₁₁ is —CN, —CH₂—NH₂, or —CH₂—NH—R₁₁″, and R₁₁″ is an amino protecting group;
R₁₂ is H, an amino protecting group, or 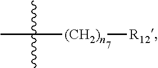
wherein R₁₂′ is —CN, —CH₂—NH₂, or —CH₂—NH—R₁₂″, and R₁₂″ is an amino protecting group;
R₁₃ is —CN, —CH₂—NH₂, or —CH₂—NH—R₁₃′, and R₁₃′ is an amino protecting group;
n₁-n₇ are independently selected from an integer of 0-10; and
n′ is 0 or 1;
the preparation method comprises a step (a) of reacting a compound of formula IV with a compound of formula V and/or a compound of formula VI: 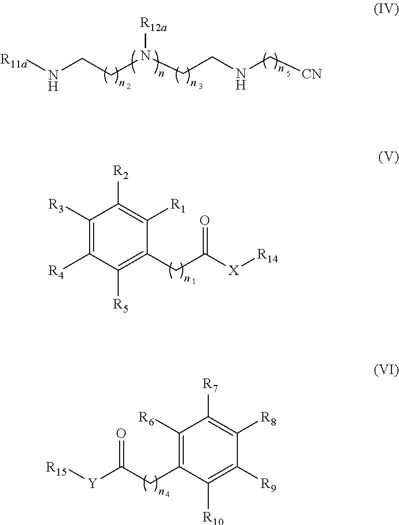
wherein R₁₁ₐ is H, an amino protecting group, or 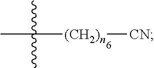
R₁₂ₐ is H, an amino protecting group, or 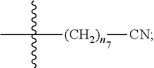
X and Y are independently selected from O and S;
R₁₄ and R₁₅ are independently selected from 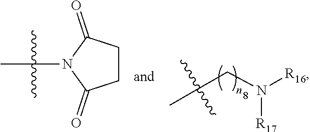
wherein n₈ is an integer of 1-6, and R₁₆ and R₁₇ are independently selected from alkyl;
and n₁-n₇, n′, and R₁-R₁₀ are as defined as in the compound of formula III;
optionally, the preparation method further comprises step (b): reducing the reaction product of step (a).

22. The preparation method according to claim 21, wherein R₁₆ and R₁₁ are independently selected from C₁-C₆ alkyl.

23. The preparation method according to claim 21, wherein R₁₄ and R₁₅ are independently selected from:

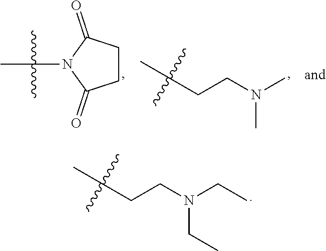

24. The preparation method according to claim 21, wherein the reaction is carried out in a solvent, wherein the solvent is selected from: ethyl acetate, isopropyl acetate, acetonitrile, dichloromethane, tetrahydrofuran, toluene, xylene, chlorobenzene, and dioxane.

25. The preparation method according to claim 24, wherein the solvent is ethyl acetate, toluene, or dioxane.

26. The preparation method according to claim 21, wherein the reaction is carried out at a temperature of 50 to 70° C.

27. The preparation method according to claim 21, wherein the compound of formula IV has the following structure: 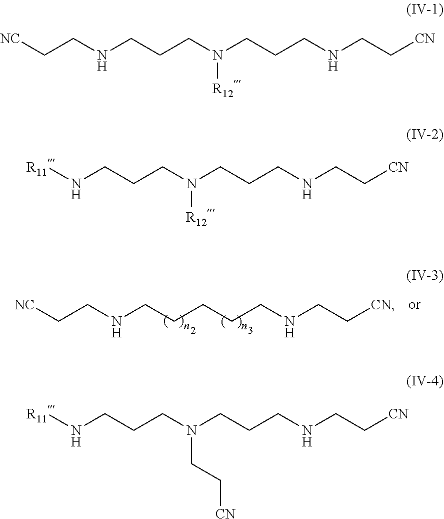
wherein R₁₁′″ and R₁₂′″ are independently selected from an amino protecting group.

28. The preparation method according to claim 21, wherein R₂, R₃, R₇, and R₈ are independently selected from: H, OH, C₁-C₆ alkoxy, C₆-C₁₂ aryloxy, and C₇-C₁₂ aralkoxy.

29. The preparation method according to claim 21, wherein R₁₁ is 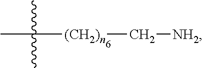
R₁₂ is H, and R₁₃ is —CH₂—NH₂, and the preparation method further comprises step (c): subjecting the reduced product of step (b) to an amino deprotection treatment.

30. The preparation method according to claim 21, wherein the compound of formula III has the following structure: [chemical structure]
